(19) United States
(12) Patent Application Publication
(10) Pub. No.: US 2017/0042785 A1
(43) Pub. Date: Feb. 16, 2017

(54) PROCESS FOR STYLING THE HAIR WITH A POLYVINYLPYRIDINE

(71) Applicant: L'OREAL, Paris (FR)

(72) Inventor: Gwenaëlle JEGOU, Saint Michel sur Orge (FR)

(21) Appl. No.: 15/303,811

(22) PCT Filed: Apr. 13, 2015
(86) PCT No.: PCT/EP2015/057931

§ 371 Date: Oct. 13, 2016

(30) Foreign Application Priority Data
Apr. 14, 2014 (FR) 1453307

(51) Int. Cl. A61K 8/81; A61Q 5/06; A45D 7/04; A61Q 5/02

(52) U.S. Cl. CPC A61K 8/817; A61Q 5/02; A61Q 5/06; A45D 7/04; A61K 2800/5422; A61K 2800/884; A45D 2007/008

(57) ABSTRACT

The invention relates to a hairstyling process comprising the following steps:

the application to the hair of a first cleansing cosmetic composition comprising one or more surfactants;
the application of a second aqueous cosmetic composition comprising a neutralized non-crosslinked vinylpyridine polymer;
followed by a step of rinsing with water and then a step of shaping the hair, and then a step of drying the hair.

The invention also relates to a kit comprising a first cleansing cosmetic composition comprising one or more surfactants and a second aqueous cosmetic composition comprising a neutralized non-crosslinked vinylpyridine polymer, the first and second compositions each being packaged in a separate packaging assembly.

The invention relates to a process for shaping the hairstyle using an aqueous composition comprising a vinylpyridine polymer and a cleansing composition (shampoo).

In the field of hairstyling, in particular for products intended for shaping and/or holding the hairstyle, hair compositions are generally in the form of hair gels, lotions, mousses or sprays. Hairstyling gels are generally water-based or aqueous-alcoholic gels containing fixing polymers and gelling agents; they are applied with the fingers onto dry or wet hair. After applying to the hair, the gels form deposits in non-uniform packets which often have a tacky feel that fluctuates as a function of the ambient humidity: the hairstyling effect is therefore not uniform over the entire head of hair.

Moreover, hairstyling gels generally leave, after styling, a tactile sensation of residual products in the hands, especially a phenomenon of residual tackiness which remains on the hands, making it necessary to wash the hands after applying these compositions.

The aim of the present invention is thus to have available a hairstyling process that is quick to perform, for obtaining good hair fixing and hold properties, while at the same time conserving a clean and non-tacky feel of the hair and which may optionally be used directly during showering or shampooing.

The Applicant has demonstrated that by performing a hairstyling process comprising the application of an aqueous composition comprising a vinylpyridine polymer and a cleansing composition (shampoo) as defined below, it is possible to obtain hairstyling that shows good fixing properties. In particular, the process makes it possible to style the hair with a novel uniform and non-tacky lock spiking effect. The process is particularly suitable for use directly during showering or shampooing. It is quick to perform.

The term “lock spiking” means that the treated hair is in the form of fine locks regularly distributed over the surface of the scalp.

A subject of the present invention is thus a hairstyling process, comprising the following steps:

the application to the hair of a first cleansing cosmetic composition comprising one or more surfactants;
the application of a second aqueous cosmetic composition comprising a vinylpyridine polymer as defined below,
followed by a step of rinsing with water and then a step of shaping the hair, and then a step of drying the hair.

A subject of the invention is also a kit comprising:

a first cleansing cosmetic composition comprising one or more surfactants and a second aqueous cosmetic composition comprising a vinylpyridine polymer as defined below, the first and second compositions each being packaged in a separate packaging assembly.

The composition packaging assembly is, in a known manner, any packaging that is suitable for storing cosmetic compositions (especially a bottle, tube, spray bottle or aerosol bottle).

Such a kit allows the hairstyling process according to the invention to be performed.

In the process according to the invention, the first and second compositions are separate.

The vinylpyridine polymer used in the process according to the invention is a neutralized non-crosslinked ethylenic polymer derived from the polymerization of:

(A) from 5% to 100% by weight of a vinylpyridine monomer (Ia) and/or (Ib):

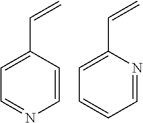

(b) 0 to 95% by weight of at least one additional nonionic or cationic ethylenic monomer chosen from:
optionally hydroxylated C₁₋₁₂ alkyl (meth)acrylates;
(meth)acrylamides bearing a group NH₂ or NHR, R being a C₁-C₁₂ alkyl group, or NR₁R₂, R₁ and R₂ being a C₁-C₁₂ alkyl group such that the total sum of the carbon number of R₁+R₂ is less than or equal to 18;
vinyl esters R′—COO—CH═CH₂, R′ being a C₁-C₁₈ alkyl radical;
vinyl ethers R″—O—CH═CH₂, R″ being a C₁-C₁₈ alkyl radical;
olefins, in particular C₂-C₂₀ alpha-olefins such as ethylene, propylene, butadiene, 1-octene, 1-butene, 1-hexadecene, 1-octadecene or eicosene;
vinyl alcohol, vinylpyrrolidone, vinylcaprolactam, N-vinylformamide, N-methyl-N-vinylformamide; N-methyl-N-vinylacetamide; vinyl butyral; vinyl carbazole; vinyl chloride; vinylidene chloride (or 1,1-dichloroethylene), cis-isoprene, trans-isoprene; vinylaromatic monomers containing from 8 to 20 carbon atoms and better still containing from 8 to 14 carbon atoms. Such monomers are, for example, styrene, 1-vinylnaphthalene, 2-vinylnaphthalene, 2-methylstyrene (or ortho-methylstyrene), 3-methylstyrene, 4-methylstyrene (or para-methylstyrene), 4-propylstyrene, 4-cyclohexylstyrene, 4-dodecylstyrene, 2-ethyl-4-benzylstyrene, 4-(phenylbutyl)styrene, 4-tert-butylstyrene, 4-acetoxystyrene, 4-methoxystyrene, 4-methylstyrene, 2-chlorostyrene, 3-chlorostyrene, 4-chlorostyrene, dimethylstyrene, 2,6-dichlorostyrene, 2,4-dimethylstyrene; 2,5-dimethylstyrene, 3,5-diethoxystyrene, 2,4-diethoxystyrene, 4-fluorostyrene. Use may preferably be made of styrene, para-methylstyrene or ortho-methylstyrene, and mixtures thereof. The vinylaromatic monomer that is particularly preferred is styrene;
and mixtures thereof;
the polymer not containing any anionic monomer;
said polymer being at least 10% neutralized.

Monomer (Ia) is 4-vinylpyridine and monomer (Ib) is 2-vinylpyridine.

The additional ethylenic monomer is preferably chosen from styrene, butyl methacrylate, 2-hydroxyethyl methacrylate, lauryl methacrylate, ethylene, propylene and butadiene, and mixtures thereof.

The vinylpyridine monomer is at least 10%, especially at least 20%, or at least 30%, or at least 40%, or at least 50% neutralized, or even totally neutralized. The neutralized form makes it possible to obtain a polymer that is soluble or dispersible in water (at 25° C.) and thus to be suitable for use in the process in a rinse-out mode.

The degree of neutralization corresponds to the percentage of the number of neutralized vinylpyridine units relative to the total number of vinylpyridine units present in the polymer.

The neutralization of the vinylpyridine polymer may be performed by addition of acid, especially of one or more acids.

Use may be made of mineral acids, such as sulfuric acid, hydrochloric acid, hydrobromic acid, hydriodic acid or phosphoric acid.

Use may also be made of organic acids, which may especially comprise one or more carboxylic, sulfonic or phosphonic acid groups. They may be linear, branched or cyclic aliphatic acids, or alternatively aromatic acids. These acids may also include one or more heteroatoms chosen from O and N, for example in the form of hydroxyl groups. Mention may be made especially of propionic acid, acetic acid, lactic acid, terephthalic acid, citric acid and tartaric acid, the acid of formula HO₂C—CH₂(CH₂CH₂O)ₙCH₂—CO₂H with n ranging from 4 to 20, betaine HCl [(CH₃)₃N+CH₂CO₂H.Cl—], gluconic acid, 2-ethylcaproic acid, oleic acid, behenic acid, stearic acid, glutamic acid and aspartic acid.

Preferentially, the vinylpyridine polymer is neutralized with hydrochloric acid.

If the vinylpyridine polymer is not neutralized, no lock spiking effect may be obtained by using such a polymer with the process steps defined previously.

Preferably, the vinylpyridine polymer may be chosen from poly(4-vinylpyridinium) chloride, poly(2-vinylpyridinium) chloride, poly(4-vinylpyridinium) chloride-co-styrene; poly(2-vinylpyridinium) chloride-co-styrene, poly(4-vinylpyridinium) chloride-co-butyl methacrylate, poly(4-vinylpyridinium) chloride-co-butadiene; poly(2-vinylpyridinium) chloride-co-butadiene.

Preferentially, the vinylpyridine polymer may be chosen from 2-vinylpyridine homopolymer and 4-vinylpyridine homopolymer.

Advantageously, the content of 2-vinylpyridine and/or 4-vinylpyridine monomers ranges from 50% to 100%, preferably ranges from 55% to 100% and preferentially ranges from 60% to 100% of the total weight of monomers. These monomer contents are used to obtain, after polymerization, the vinylpyridine polymer described previously.

The vinylpyridine polymer may be a linear or branched polymer. It is preferably linear. This polymer may be a statistical, sequenced, block or gradient polymer. Advantageously, it is in the form of a statistical polymer.

The neutralized polyvinylpyridine polymer used according to the invention may preferably be conveyed in aqueous medium, i.e. it is preferably water-soluble or water-dispersible.

The term “water-soluble” means that it is soluble in water to a proportion of at least 1% by weight, at 25° C., and forms a clear solution.

The term “water-dispersible” means that it forms in water, at a concentration of 1% by weight, at 25° C., a stable dispersion of generally spherical, fine particles.

The mean size of the particles constituting said dispersion is less than 1 μm, and more generally ranges between 5 and 400 nm, preferably from 10 to 250 nm.

These particle sizes may be measured by light scattering.

Preferably, the vinylpyridine polymer has a weight-average molecular weight ranging from 1000 to 300 000 g/mol and preferably ranging from 1000 to 200 000 g/mol.

Advantageously, when the vinylpyridine polymer is a homopolymer, it preferably has a molecular weight ranging from 1000 to 20 000 g/mol.

The vinylpyridine polymer may be present in the aqueous composition in a content ranging from 0.5% to 20% by weight, preferably ranging from 1% to 15% by weight and more preferentially ranging from 5% to 15% by weight, relative to the total weight of the composition.

The composition comprising the vinylpyridine polymer (known as the second composition) comprises water, especially in a content ranging from 50% to 99.5% by weight and preferably ranging from 60% to 99% by weight, relative to the total weight of the composition.

Advantageously, the second composition does not contain any anionic surfactant.

The term “cosmetic composition” is understood to mean a composition that is compatible with keratin materials, which has a pleasant colour, odour and feel and which does not cause unacceptable discomfort (stinging, tautness or redness) liable to discourage the consumer from using it.

The term “keratin materials” is understood to mean the skin (body, face, contour of the eyes, scalp), head hair, eyelashes, eyebrows, bodily hairs, nails or lips.

The process according to the invention uses a cleansing composition comprising one or more surfactants (known as the first composition).

The detergent surfactant may be chosen from anionic surfactants, nonionic surfactants and amphoteric surfactants, and mixtures thereof.

The term “anionic surfactant” means a surfactant comprising, as ionic or ionizable groups, only anionic groups. These anionic groups are preferably chosen from the following groups:

—C(O)OH, —O(O)O—, —SO₃H, —S(O)₂O —, —OS(O)₂OH, —OS(O)₂O —, —P(O)OH₂, —P(O)₂O—, —P(O)O₂—, —P(OH)₂, ═P(O)OH, —P(OH)O—, ═P(O)O—, ═POH, ═PO—, the anionic parts comprising a cationic counterion such as an alkali metal, an alkaline earth metal or an ammonium.

As examples of anionic surfactants that may be used in the composition according to the invention, mention may be made of alkyl sulfates, alkyl ether sulfates, alkylamido ether sulfates, alkylaryl polyether sulfates, monoglyceride sulfates, alkylsulfonates, alkylamidesulfonates, alkylarylsulfonates, α-olefin sulfonates, paraffin sulfonates, alkyl sulfosuccinates, alkyl ether sulfosuccinates, alkylamide sulfosuccinates, alkyl sulfoacetates, acylsarcosinates, acylglutamates, alkyl sulfosuccinamates, acylisethionates and N-acyltaurates, polyglycoside-polycarboxylic acid and alkyl monoester salts, acyl lactylates, salts of D-galactoside uronic acids, salts of alkyl ether carboxylic acids, salts of alkylaryl ether carboxylic acids, salts of alkylamido ether carboxylic acids; and the corresponding non-salified forms of all these compounds; the alkyl and acyl groups of all these compounds comprising from 6 to 40 carbon atoms and the aryl group denoting a phenyl group. These compounds can be oxyethylenated and then preferably comprise from 1 to 50 ethylene oxide units.

The salts of C₆-C₂₄ alkyl monoesters of polyglycoside-polycarboxylic acids may be chosen from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactants are in salt form, they may be chosen from alkali metal salts such as the sodium or potassium salt and preferably the sodium salt, ammonium salts, amine salts and in particular amino alcohol salts or alkaline earth metal salts such as the magnesium salts.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts. Alkali metal or alkaline earth metal salts, and in particular sodium or magnesium salts, are preferably used.

Among the anionic surfactants, (C₆-C₂₄)alkyl sulfates, (C₆-C₂₄)alkyl ether sulfates comprising from 2 to 50 ethylene oxide units, especially in the form of alkali metal, ammonium, amino alcohol and alkaline earth metal salts, or a mixture of these compounds, are most particularly preferred.

Better still, (C₁₂-C₂₀)alkyl sulfates, (C₁₂-C₂₀)alkyl ether sulfates comprising from 2 to 20 ethylene oxide units, especially in the form of alkali metal, ammonium, amino alcohol and alkaline earth metal salts, or a mixture of these compounds, are preferred. Better still, sodium lauryl ether sulfate containing from 2 to 50 (especially from 2 to 3) ethylene oxide units is preferred.

The nonionic surfactants that may be used may be chosen from alcohols, α-diols and (C₁₋₂₀)alkylphenols, these compounds being polyethoxylated, polypropoxylated or bearing a fatty chain comprising, for example, from 8 to 30 carbon atoms and especially from 16 to 30 carbon atoms, the number of ethylene oxide and/or propylene oxide groups possibly ranging especially from 2 to 50, and the number of glycerol groups possibly ranging especially from 2 to 30.

Mention may also be made of condensates of ethylene oxide and of propylene oxide with fatty alcohols; polyethoxylated fatty amides preferably having from 2 to 30 ethylene oxide units, polyglycerolated fatty amides containing on average from 1 to 5, and in particular from 1.5 to 4, glycerol groups; ethoxylated fatty acid esters of sorbitan preferably containing from 2 to 40 ethylene oxide units, fatty acid esters of sucrose, fatty acid esters of polyethylene glycol, N—(C₆₋₂₄ alkyl)glucamine derivatives, amine oxides such as (C₁₀₋₁₄alkyl)amine oxides or N—(C₁₀₋₁₄ acyl)aminopropylmorpholine oxides.

Mention may also be made of nonionic surfactants of alkylpolyglycoside type, represented by the following general formula:

R₁O—(R₂O)ₜ-(G)ᵥ

in which:
R₁ represents a linear or branched alkyl or alkenyl radical comprising 6 to 24 carbon atoms and especially 8 to 18 carbon atoms, or an alkylphenyl radical whose linear or branched alkyl radical comprises 6 to 24 carbon atoms and especially 8 to 18 carbon atoms;
R₂ represents an alkylene radical comprising 2 to 4 carbon atoms,
G represents a sugar unit comprising 5 to 6 carbon atoms,
t denotes a value ranging from 0 to 10 and preferably 0 to 4,
v denotes a value ranging from 1 to 15 and preferably 1 to 4.

Preferably, the alkylpolyglycoside surfactants are compounds having the formula described above in which R₁ denotes a saturated or unsaturated, linear or branched alkyl radical comprising from 8 to 18 carbon atoms, t denotes a value ranging from 0 to 3, preferably equal to 0, G denotes glucose, fructose or galactose, preferably glucose; the degree of polymerization, i.e. the value of v, may range from 1 to 15 and preferably from 1 to 4; the mean degree of polymerization more particularly being between 1 and 2. The glucoside bonds between the sugar units are generally of 1-6 or 1-4 type and preferably of 1-4 type.

Among the commercial products, mention may be made of the products sold by the company Cognis under the names Plantaren® (600 CS/U, 1200 and 2000) or Plantacare® (818, 1200 and 2000); the products sold by the company SEPPIC under the names Triton CG110 (or Oramix CG 10) and Triton CG312 (or Oramix® NS 10); the products sold by the company BASF under the name Lutensol GD 70, or else the products sold by the company Chem Y under the name AG10 LK. Preferably, use is made of C₈/C₁₆-alkyl polyglycosides 1,4, especially in an aqueous 53% solution, such as those sold by Cognis under the reference Plantacare® 818 UP.

The amphoteric surfactants that may be used in the invention may be optionally quaternized secondary or tertiary aliphatic amine derivatives, in which the aliphatic group is a linear or branched chain comprising from 8 to 22 carbon atoms, said amine derivatives containing at least one anionic group, for instance a carboxylate, sulfonate, sulfate, phosphate or phosphonate group.

Mention may be made in particular of (C₈-C₂₀)alkylbetaines, sulfobetaines, alkyl(C₈-C₂₀)sulfobetaines, (C₈-C₂₀)alkylamido(C₁-C₆)alkylbetaines, such as cocamidopropylbetaine, and (C₈-C₂₀)alkylamido(C₁-C₆)alkylsulfobetaines.

Among the optionally quaternized secondary or tertiary aliphatic amine derivatives that may be used, mention may also be made of the products having the following respective structures (A2) and (A3):

Rₐ—CON(Z)CH₂₋(CH₂)ₘ—N⁺(R_{b})(R_{c})(CH₂COO⁻) (A2)

in which:
Rₐ represents a C₁₀-C₃₀ alkyl or alkenyl group derived from an acid Rₐ—COOH preferably present in hydrolysed coconut oil, or a heptyl, nonyl or undecyl group,
R_{b} represents a β-hydroxyethyl group,
R_{c} represents a carboxymethyl group;
m is equal to 0, 1 or 2,
Z represents a hydrogen atom or a hydroxyethyl or carboxymethyl group,

R_{a′}—CON(Z)CH₂₋(CH₂)_{m′}—N(B)(B′) (A3)

in which:
B represents —CH₂CH₂OX′, with X′ representing —CH₂—COOH, CH₂—COOZ′, —CH₂CH₂—COOH, —CH₂CH₂—COOZ′, or a hydrogen atom,
B′ represents —(CH₂)_{z}—Y′, with z=1 or 2, and Y′ representing —COOH, —COOZ′, —CH₂—CHOH—SO₃H or —CH₂—CHOH—SO₃Z′,
m′ is equal to 0, 1 or 2,
Z represents a hydrogen atom or a hydroxyethyl or carboxymethyl group,
Z′ represents an ion resulting from an alkali metal or alkaline earth metal, such as sodium, potassium or magnesium; an ammonium ion; or an ion resulting from an organic amine and in particular from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane,
R_{a′} represents a C₁₀-C₃₀ alkyl or alkenyl group of an acid R_{a′} COOH preferably present in hydrolysed linseed oil or coconut oil, an alkyl group, in particular a C₁₇ alkyl group, and its iso form, or an unsaturated C₁₇ group.

The compounds corresponding to formula (A3) are preferred. The compounds of formulae (A2) and (A3) are also classified in the CTFA dictionary, 5th edition, 1993, under the names disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylamphodipropionate, disodium caprylamphodipropionate, lauroamphodipropionic acid, cocoamphodipropionic acid.

Examples that may be mentioned include the cocoamphodiacetate sold by the company Rhodia under the trade name Miranol® C2M Concentrate or under the trade name Miranol Ultra C 32 and the product sold by the company Chimex under the trade name Chimexane HA.

Use may also be made of compounds of formula (A4):

R_{a″}—NH—CH(Y″)—(CH₂)n—C(O)—NH—(CH₂)n′—N(R_{d})(Rₑ) (A4)

in which:
R_{a″} represents a C₁₀-C₃₀ alkyl or alkenyl group of an acid R_{a″}—C(O)OH, which is preferably present in hydrolysed linseed oil or coconut oil;
Y″ represents the group —C(O)OH, —C(O)OZ″, —CH₂—CH(OH)—SO₃H or the group —CH₂—CH(OH)—SO₃—Z″, with Z″ representing a cationic counterion resulting from an alkali metal or alkaline earth metal, such as sodium, an ammonium ion or an ion resulting from an organic amine;
R_{d} and Rₑ represent, independently of each other, a C₁-C₄ alkyl or hydroxyalkyl radical; and
n and n′ denote, independently of each other, an integer ranging from 1 to 3.
   Mention may in particular be made of the compound classified in the CTFA dictionary under the name sodium diethylaminopropyl cocoaspartamide and sold by the company Chimex under the name Chimexane HB.

Preferably, the amphoteric surfactants are chosen from (C₈-C₂₀)alkyl betaines, (C₈-C₂₀)alkylamido(C₁-C₆)alkyl betaines and (C₈-C₂₀)alkylamphodiacetates, and also the sodium salt of diethylaminopropyl laurylaminosuccinamate, and mixtures thereof.

Preferably, the cleansing composition comprises an anionic surfactant, such as those described previously.

Preferably, the cleansing composition used according to the invention comprises a total amount of surfactants ranging from 1% to 50% by weight, preferably ranging from 2% to 35% by weight and preferentially from 3% to 25% by weight, relative to the total weight of the composition.

The cleansing composition used according to the invention is preferably aqueous and then comprises water at a concentration preferably ranging from 5% to 98% by weight, in particular from 20% to 95% by weight and better still from 50% to 80% by weight, relative to the total weight of the composition.

The first and/or second composition may also comprise one or more organic solvents that are liquid at 25° C. and 1 atm. (101 325 Pa), and especially water-soluble, such as C₁-C₇ alcohols, especially C₁-C₇ aliphatic or aromatic monoalcohols, and C₃-C₇ polyols and polyol ethers, which may thus be used alone or as a mixture with water. Advantageously, the organic solvent may be chosen from ethanol and isopropanol, and mixtures thereof.

The first and second compositions used according to the invention may comprise additional cosmetic ingredients chosen in particular from preserving agents, fragrances, sunscreens, oils, waxes, thickeners, film-forming polymers, moisturizers, vitamins, proteins, ceramides, antioxidants, agents for combating free radicals, dyestuffs, antidandruff agents, chelating agents, propellants and fillers.

Needless to say, a person skilled in the art will take care to select this or these optional additional compound(s), and/or the amount thereof, such that the advantageous properties of the composition according to the invention are not, or are not substantially, adversely affected by the envisaged addition.

According to a first embodiment of the process according to the invention (preferred mode), the following are applied, in this order:

the second cleansing cosmetic composition without performing rinsing, and then the first aqueous cosmetic composition comprising the vinylpyridine polymer,
the mixture is then left to act, especially for 10 seconds to 10 minutes,
and it is then rinsed out with water. The second cleansing composition may be worked into a lather after it has been applied to the hair or after the first composition has been applied.

According to a second embodiment of the process according to the invention, the following are applied, in this order:

the first aqueous cosmetic composition comprising the vinylpyridine polymer, without performing rinsing,
and the second cleansing cosmetic composition is then applied,
and the mixture is then left to act, especially for 10 seconds to 10 minutes,
and it is then rinsed out with water. The second cleansing composition is worked into a lather after it has been applied to the hair.

After rinsing with water, the hair is shaped, especially with the fingers or a comb or a brush, and then dried. The drying step may be performed at a temperature ranging from 20° C. to 80° C. In practice, the drying step may take place either at room temperature or using a hairdryer.

Advantageously, the process is applied to wet hair.

If the first and second compositions are mixed together before applying them to the hair, the mixture is inhomogeneous and, during rinsing, the neutralized poly(4-vinylpyridine) polymer is removed and is not deposited on the hair in sufficient amount to be able to obtain a lock spiking effect. Under these conditions, no lock spiking effect may be obtained by applying to the hair the mixture of shampoo and of neutralized poly(4-vinylpyridine). It is thus necessary to perform separate application to the hair of the first and second compositions in order to obtain the lock spiking effect.

The examples hereinafter illustrate the invention without, however, limiting the scope thereof. The amounts of the ingredients indicated are expressed as weight percentages.

Aqueous compositions of 6 polymers neutralized according to the invention were prepared by mixing the vinylpyridine polymer and the neutralizing agent 1N HCl and adding water. The neutralization is performed to a degree of 50%.

### EXAMPLE 1

10 g of poly(4-vinylpyridine) Mw 60 000 (reference 472344 from Aldrich) were mixed with 47 ml of 1N HCl, and the mixture was then made up to 100 ml with water. A yellow liquid aqueous solution was obtained.

### EXAMPLE 2

In the same manner as in Example 1, 10 g of poly(4-vinylpyridine) Mw 160 000 (reference 472352 from Aldrich) were mixed with 47 ml of 1N HCl, and the mixture was then made up to 100 ml. A yellow liquid aqueous solution was obtained.

### EXAMPLE 3

In the same manner as in Example 1, 10 g of 4-vinylpyridine/styrene copolymer (reference 192074 from Aldrich) were mixed with 35 ml of 1N HCl, and the mixture was then made up to 100 ml. A pale yellow liquid aqueous solution was obtained.

### EXAMPLE 4

In the same manner as in Example 1, 10 g of 4-vinylpyridine/butyl methacrylate (90/10) copolymer (reference 306258 from Aldrich) were mixed with 42 ml of 1N HCl, and the mixture was then made up to 100 ml. A yellow liquid aqueous solution was obtained.

### EXAMPLE 5

In the same manner as in Example 1, 10 g of poly(2-vinylpyridine) Mw 11 000 (reference 523305 from Aldrich) were mixed with 47 ml of 1N HCl, and the mixture was then made up to 100 ml. A yellow liquid aqueous solution was obtained.

### EXAMPLE 6

In the same manner as in Example 1, 20 g of a 50% AM solution of poly(4-vinylpyridine) Mw 7000 in methanol (Reilline 410 from Vertellus) were mixed with 47 ml of 1N HCl, and the mixture was then made up to 100 ml. The methanol was evaporated off on a rotary evaporator. An orange liquid aqueous solution was obtained.

### Comparative Examples 7 to 14 Outside the Invention

Aqueous solutions containing 10% by weight of polymer were also prepared with the following polymers outside the invention (compositions C1 to C14):

| | | Concentration |
|---|---|---|
| Example | Polymers | in water |
| 7 | Poly(2-vinylpyridine) 2% crosslinked | 10% |
| | with divinylbenzene (reference 547689 | |
| | from Aldrich), 50% neutralized with HCl | |
| 8 | Vinylamine/N-vinylformamide copolymer | 10% |
| | (Luviquat ® 9030 from BASF) | |
| 9 | Polydimethyldiallylammonium chloride | 10% |
| | (Merquat ® 100 from Nalco) | |
| 10 | Hydroxypropyl guar trimethylammonium | 10% |
| | chloride (Jaguar ® C-13S from Rhodia) | |
| 11 | Vinylpyrrolidone/dimethylaminoethyl | 10% |
| | methacrylate copolymer (Copolymer | |
| | 845-0 from ISP) | |
| 12 | 1-Vinyl-2- | 10% |
| | pyrrolidone/methacrylamide/1- | |
| | vinylimidazole terpolymer (Luviset ® | |
| | Clear from BASF) | |
| 13 | Vinylpyrrolidone/vinylimidazole (50/50) | 10% |
| | copolymer (Luvitec VPI 55K72W from | |
| | BASF) | |
| 14 | Vinyl acetate/vinyl p-tert- | 10% |
| | butylbenzoate/crotonic acid (65/25/10) | |
| | copolymer (Mexomer PW from Chimex) | |
| | 50% neutralized with 2-amino-2-methyl- | |
| | 1-propanol | |

### Shampoo Composition:

The shampoo sold under the name DOP Camomile by the company Lascad, which contains sodium lauryl ether sulfate (2 ethylene oxide units), was used.

Evaluation of the Hairstyling Properties Obtained with the Hairstyling Processes Using Compositions C1 to C14:

The following treatment process was performed with each of the compositions of Examples 1 to 6 in accordance with the invention and each of the compositions of the Comparative Examples 7 to 14:

1 g of shampoo and then 2 g of the polymer composition were applied to locks of wet hair (2.7 g). The impregnated lock of hair was massaged for 20 seconds. The mixture was left to act at room temperature for 3 minutes, and the locks were then rinsed and dried under a hood at 45° C.

A panel of five judges was then asked to evaluate the feel of the hair.

### Grading:

Feel: 0 (very poor) to 5 (excellent)

After 5 hours at room temperature and ambient humidity, the same judges evaluated the lock spiking effect (for excellent lock spiking on a lock, the hairs are stuck together, the lock has set to a solid and forms a stick).
Wear property: 0 (very poor) to 5 (excellent)

| | | Hairstyling |
|---|---|---|
| | Feel | effect |
| Shampoo alone | 5 | 0 |
| Example 1 | 4 | 4 |
| Example 2 | 3.5 | 4.5 |
| Example 3 | 3.5 | 3.5 |
| Example 4 | 3.5 | 3.5 |
| Example 5 | 4.5 | 4 |
| Example 6 | 4.5 | 4.5 |
| Comparative 1 | 4 | 1.5 |
| Comparative 2 | 2.5 | 0.5 |
| Comparative 3 | 4.5 | 0.5 |
| Comparative 4 | 4.5 | 0 |
| Comparative 5 | 3.5 | 0.5 |
| Comparative 6 | 3.5 | 0.5 |
| Comparative 7 | 3.5 | 0 |
| Comparative 8 | 3 | 0 |
| Comparative 9 1 | 3 | 1 |

The results obtained show that only the processes using compositions C1 to C6 according to the invention make it possible to obtain a good lock spiking effect. For these compositions, the lock spiking effect was also evaluated on a malleable head according to the following protocol:

10 g of shampoo were deposited on the wet malleable head, and 10 g of the composition to be evaluated (composition C1 to C6) were then applied to half of the malleable head. Each of the two half-heads were massaged for 2 minutes and then rinsed with water. The hair was shaped with a comb and then dried under a hood at 45° C. The lock spiking obtained (appearance of the hairstyle) was then evaluated.

Quality of the lock spiking (appearance of the hairstyle): 0 (very poor) to 5 (excellent)

| | Quality of |
|---|---|
| | the lock |
| | spiking |
| Shampoo alone | No lock |
| | spiking |
| Example 1′ | 3.5 |
| Example 2′ | 4 |
| Example 3′ | 3.5 |
| Example 4′ | 4 |
| Example 5′ | 4.5 |
| Example 6′ | 4.5 |

The treatment processes in accordance with the invention (Example 1′ to 6′) make it possible to obtain a novel lock spiking effect.

### Comparative Example 15 Outside the Invention

Instead of performing the treatment process in two separate steps, the aqueous solution of poly(4-vinylpyridine) of Example 1 was premixed with the DOP Camomile shampoo (50/50 weight mixture). The mixture is inhomogeneous. The mixture was then applied to a lock of wet hair, according to the protocol described previously.

During rinsing, the poly(4-vinylpyridine) polymer is removed and is not deposited on the hair in sufficient amount to be able to obtain a lock spiking effect. Under these conditions, no lock spiking effect may be obtained by applying to the hair the mixture of shampoo and of poly(4-vinylpyridine).

### Comparative Example 16 Outside the Invention

The initial polymer of Example 1 was used without performing neutralization. The polymer is insoluble and not dispersible in water. It remains precipitated in the water and, in this form, cannot be used in the hairstyling process. Under these conditions, no lock spiking effect can be obtained with the non-neutralized polymer.

1. Hairstyling process comprising the following steps:
the application to the hair of a first cleansing cosmetic composition comprising one or more surfactants;
the application of a second aqueous cosmetic composition comprising a vinylpyridine polymer, which is a neutralized non-crosslinked ethylenic polymer derived from the polymerization of:
(A) from 5% to 100% by weight of a vinylpyridine monomer (Ia) and/or (Ib): 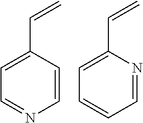
(b) 0 to 95% by weight of at least one additional nonionic or cationic ethylenic monomer chosen from:
optionally hydroxylated C₁₋₁₂ alkyl (meth)acrylates;
(meth)acrylamides bearing a group NH₂ or NHR, R being a C₁-C₁₂ alkyl group, or NR₁R₂, R₁ and R₂ being a C₁-C₁₂ alkyl group such that the total sum of the carbon number of R₁+R₂ is less than or equal to 18;
vinyl esters R′—COO—CH═CH₂, R′ being a C₁-C₁₈ alkyl radical;
vinyl ethers R″—O—CH═CH₂, R″ being a C₁-C₁₈ alkyl radical;
olefins, in particular C₂-C₂₀ alpha-olefins;
vinyl alcohol, vinylpyrrolidone, vinylcaprolactam, N-vinylformamide, N-methyl-N-vinylformamide; N-methyl-N-vinylacetamide; vinyl butyral; vinyl carbazole; vinyl chloride; vinylidene chloride (or 1,1-dichloroethylene), cis-isoprene, trans-isoprene;
vinylaromatic monomers containing from 8 to 20 carbon atoms and better still containing from 8 to 14 carbon atoms;
and mixtures thereof;
the polymer not containing any anionic monomer;
said polymer being at least 10% neutralized;
followed by a step of rinsing with water and then a step of shaping the hair, and then a step of drying the hair.

2. Process according to the preceding claim, in which the first composition comprises one or more surfactants chosen from anionic surfactants, nonionic surfactants and amphoteric surfactants, and mixtures thereof, and preferably from anionic surfactants.

3. Process according to either of the preceding claims, in which the first composition comprises a total amount of surfactants ranging from 1% to 50% by weight, preferably ranging from 2% to 35% by weight and preferentially from 3% to 25% by weight, relative to the total weight of the composition.

4. Process according to the preceding claim, in which the additional ethylenic monomer is preferably chosen from styrene, butyl methacrylate, 2-hydroxyethyl methacrylate, lauryl methacrylate, ethylene, propylene and butadiene, and mixtures thereof.

5. Process according to one of the preceding claims, in which the vinylpyridine polymer is neutralized with one or more acids, chosen especially from sulfuric acid, hydrochloric acid, hydrobromic acid, hydriodic acid, phosphoric acid, propionic acid, acetic acid, lactic acid, terephthalic acid, citric acid and tartaric acid, the acid of formula HO₂C—CH₂(CH₂CH₂O)ₙCH₂—CO₂H with n ranging from 4 to 20, betaine HCl [(CH₃)₃N+CH₂CO₂H.Cl⁻], gluconic acid, 2-ethylcaproic acid, oleic acid, behenic acid, stearic acid, glutamic acid and aspartic acid, and preferably hydrochloric acid.

6. Process according to one of the preceding claims, in which the vinylpyridine polymer is chosen from poly(4-vinylpyridinium) chloride, poly(2-vinylpyridinium) chloride, poly(4-vinylpyridinium) chloride-co-styrene; poly(2-vinylpyridinium) chloride-co-styrene, poly(4-vinylpyridinium) chloride-co-butyl methacrylate, poly(4-vinylpyridinium) chloride-co-butadiene; poly(2-vinylpyridinium) chloride-co-butadiene.

7. Process according to one of the preceding claims, in which the vinylpyridine polymer is chosen from 2-vinylpyridine homopolymer and 4-vinylpyridine homopolymer.

8. Process according to one of claims 1 to 6, in which the content of 2-vinylpyridine and/or 4-vinylpyridine monomers ranges from 50% to 100%, preferably ranges from 55% to 100% and preferentially ranges from 60% to 100% of the total weight of monomers of the vinylpyridine polymer.

9. Process according to one of the preceding claims, in which the neutralized vinylpyridine polymer is water-soluble or water-dispersible.

10. Process according to one of the preceding claims, in which the vinylpyridine polymer has a weight-average molecular weight ranging from 1000 to 300 000 g/mol, preferably ranging from 1000 to 200 000 g/mol and especially, when the vinylpyridine polymer is a homopolymer, ranging from 1000 to 20 000 g/mol.

11. Process according to one of the preceding claims, in which the vinylpyridine polymer is present in the aqueous composition in a content ranging from 0.5% to 20% by weight, preferably ranging from 1% to 15% by weight and more preferentially ranging from 5% to 15% by weight relative to the total weight of the composition.

12. Process according to one of the preceding claims, in which the second composition does not contain any anionic surfactant.

13. Process according to one of the preceding claims, in which the following are applied to the hair, in this order:
the second cleansing cosmetic composition without performing rinsing,
and then the first aqueous cosmetic composition comprising the vinylpyridine polymer,
the mixture is then left to act, especially for 10 seconds to 10 minutes,
it is then rinsed out with water,
and the hair is then shaped and dried.

14. Process according to one of the preceding claims, in which the following are applied, in this order:
the first aqueous cosmetic composition comprising the vinylpyridine polymer, without performing rinsing,
and the second cleansing cosmetic composition is then applied,
and the mixture is then left to act, especially for 10 seconds to 10 minutes,
and it is then rinsed out with water,
and the hair is then shaped and dried.

15. Kit comprising:
a first cleansing cosmetic composition comprising one or more surfactants as defined in one of claims 1 to 3 and a second aqueous cosmetic composition comprising a vinylpyridine polymer as defined in one of claims 1 and 4 to 12, the first and second compositions each being packaged in a separate packaging assembly.
